(19) United States
(12) Patent Application Publication
(10) Pub. No.: US 2003/0068617 A1
(43) Pub. Date: Apr. 10, 2003

(54) Method for predicting regulatory elements in repetitive sequences using transcription factor binding sites

(76) Inventors: Jorng-Tzong Horng, Chung-Li (TW); Wen-Fu Chao, Guei-Shan Shiang (TW)

Correspondence Address: J.C. Patents, Inc., Suite 114, 1340 Reynolds Ave., Irvine, CA, 92614, US

(21) Appl. No.: 09/829,291

(22) Filed: Apr. 9, 2001

(51) Int. Cl. C12Q 1/68; G06F 19/00

(52) U.S. Cl. 435/6; 702/20

(57) ABSTRACT

Repeat sequences are the most abundant in the extragenic region of genomes, while a large number of regulatory elements are found in this region. The invention attempts to mine rules on how combinations of individual binding sites are distributed in repeat sequences. These mined association rules would facilitate identifying gene classes regulated by similar mechanisms and accurately predicting regulatory elements. Herein, the combinations of transcription factor binding sites in the repeat sequences are obtained, and data mining techniques are applied to mine the association rules from the combinations of binding sites. In addition, the associations are further pruned to remove insignificant associations and obtain a set of discovered associations. The discovered association rules are used to partially classify the repeat sequences in the repeat sequence database.

### BACKGROUND OF THE INVENTION

1. Field of Invention

The present invention relates to a method for mining association rules from combinations of transcription factor binding sites in repeat sequences. More particularly, the present invention relates to a method for predicting regulatory elements in repetitive sequences using transcription factor binding sites.

2. Description of Related Art

As an increasing number of genomes have been sequenced, it has ushered the study of sequences. In this area, repetitive sequences have received considerable interest. Repetitive sequences are a large amount of subsequences continuously appearing in a sequence, from two to hundred of times. Repetitive sequences are the most abundant ones in extragenic region of genome, in which a large number of regulatory elements are located. These repeats may significantly affect the chromatin structure formation in nucleus and also provide valuable insight into genetic evolution and phylogeny. Normally, the repetitive sequences whose length extends from twenty to several thousands in the genomes are in the main interest. A repeat sequence database has been constructed for repetitive sequences.

TRANSFAC is the most complete database for transcription factor binding sites and well maintained. Though consensus patterns or nucleotide distribution matrices can be used to describe transcription factor binding sites, we describe the binding sites using consensus patterns herein.

To face a large among of repeat sequences, data mining plays a prominent role in knowledge extraction. The idea of mining association rules over basket data has been introduced. An example of an association rule is given below. The work stated “50% of transactions that contain beer also contain diapers; 5% of all transactions contain both of these items”. Where 50% is called the confidence of the rule, and 5% is the support of the rule. Data mining is crucial for extracting knowledge in a database. Frequently used data mining approaches include association rules, statistical, neural network, and genetic algorithms.

In statistics, the Chi-square test (χ²) is extensively applied for testing independence and correlation. The Chi-square is based on comparing observed frequencies with the corresponding expected frequencies. That the observed frequencies are closer to the expected frequencies implies a greater weight in favor of independence. Let ƒ₀ be an observed frequency, and ƒ is an expected frequency, The Chi-square test is used to test the significance of the deviation from the expected values. The χ² value is defined as follows:

$${χ}^{2} = \sum \frac{{( {f}_{0} - f )}^{2}}{f}$$

where χ² value of 0 implies that the sites are statistically independent. If it is higher than a certain threshold value, e.g., 4.12 at the 97% significance level, we reject the independent assumption and classify it as correlated.

Previous researches of partial classification using association rules focus on identifying characteristics of some of the data classes, but fail to predict future values.

### SUMMARY OF THE INVENTION

The present invention identifies the combinations of transcription factor binding sites in repeat sequences. Data mining techniques are then applied to mine the associations from the combinations of transcription factor binding sites that occur in repeat sequences. The data mining technique can mine an enormous number of associations. The associations are then pruned, so that the insignificant ones are removed and a set of useful associations are left. In addition, the discovered associations are used to partially classify the repeat sequences in our repeat sequence database.

In this invention, combinations of transcription factor binding sites are found in the repeat sequences in a repeat sequence database. Each repeat sequence is mapped to a transaction and combinations of transcription factor binding sites are mapped to items of a transaction. The transcription factor binding sites in TRANSFAC database need to be preprocessed due to their complex characteristics. The data mining approaches, such as, Apriori and AprioriTid, are then applied to mine the associations from the combinations of transcription factor binding sites in repeat sequences. Chi-square significance level is used to remove insignificant association rules from the huge collection of generated association rules. The redundant rules are pruned and the remaining rules are classified into cover and non-cover sets. The mined rules can also be used to find useful genes in complete genomes as well as partially cluster the repeat sequences in the repeat sequence database.

The present invention develops a general software tool to find and analyze combinations of transcription factor binding sites that occur often in regions for various genomes. In addition to analyzing the association rules for the combinations, the occurrence ratios of the association rules in the genome are identified. This tool can find all the combinations satisfying the given parameters with respect to a given set of regions, its counter-set, and the chosen set of sites.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is a flow chart illustrating the proposed approach according to one preferred embodiment of this invention; and

FIG. 2 is an illustrative example of a mapping between a repeat sequence and its combinations of the transcription factor binding sites according to one preferred embodiment of this invention;

FIG. 3 is a flow chart illustrating steps of pruning and structuring according to one preferred embodiment of this invention;

FIG. 4 illustrates the partial classification rules for the Human Chromosome 22 according to one preferred embodiment of this invention;

FIG. 5 illustrates the partial classification rules for the C. Elegans Genome according to one preferred embodiment of this invention;

FIG. 6 is a schematic view of a computerized system for mining association rules from combinations of transcription factor binding sites in repeat sequences and for further predicting regulatory elements in repetitive sequences using transcription factor binding sites according to one preferred embodiment of this invention.

### DESCRIPTION OF THE PREFERRED EMBODIMENTS

TRANSFAC database (release 4.0) is the most complete database for transcription factor binding sites, which is open to public. TRANSFAC database contains 4965 site sequences and 2837 factor entries, while most sites are also consensus patterns. The TRANSFAC data can be a transcription factor binding site accession number having different consensus sequences or different binding site accession numbers having a same consensus sequence. Wild characters, such as ‘M’ or ‘W’ used in TRANSFAC, make the sequences cover a range of sequences. Small consensus sequences may appear in larger ones. A preprocessing process is required because complex characteristics of the transcription factor binding sites in TRANSFAC have to be considered.

Properties of Repeat Sequences in the Repeat Sequence Database

Repeat sequences in the repeat sequence database can be categorized as the following types:

1. Minisatellite repeats: Variable number tandem repeat (VNTR). Each repeat sequence of this type has a length ranging from ten to sixty base pairs. This repeat repeatedly appears from five to fifty times in a sequence.

2. Microsatellite repeats: Each repeat of this type has a length ranging from one to four base pairs unit repeated 10-20 times.

3. Interspersed genome-wide repeats.

Short Interspersed Nuclear Elements (SINEs): The length of each repeat is less than 280 base pairs. Repeats are repeatedly appeared in genes.

Long Interspersed Nuclear Elements (LINEs): The length of each repeat ranges from 6 to 8k base pairs. They repeatedly appear from 50,000 to 100,000 times.

4. Inverted repeats: Repeat sequences invert each other. For example, the following two repeat sequences are inverted.

5′ GATTC---GAATC 3′
3′ CTAAG---CTTAG 5′

The repeat sequences in our experiments include direct and inverted repeats whose length is larger than or equal twenty base pairs.

Properties of the Data in TRANSFAC

Genome sequences are a string of A, C, G or T. However, sequences may also be expressed in symbols (wild characters) as following:

W: A or T
R: A or G
K: G or T
B: C, G, or T
H: A, C, or T
N: A, C, G, or T
S: C or G
Y: C or T
M: A or C
D: A, G, or T
V: A, C, or G

Several examples are listed to illustrate properties of the data in TRANSFAC as followings:

### EXAMPLE 1

| | |
|---|---|
| MATWAAT | R04327 |

This example indicates that all sequences including, AATAAAT, CATAAAT, AATTAAT, CATTAAT, are designated to a same binding site identification.

### EXAMPLE 2

| | |
|---|---|
| R00018 | TGCCCTAA |
| R00018 | TGCCCTTG |
| R00018 | TGCCTGG |
| R00018 | TGGCAAAC |

Example 2indicates that site R00018 has four different binding site consensus sequences. In TRANSFAC database, 71 binding site identifications belong to this type.

### EXAMPLE 3

| | |
|---|---|
| R01372 | GGGGC |
| R01241 | GGGGC |
| R01243 | GGGGC |

Example 3 indicates different binding sites having the same consensus sequence.

### EXAMPLE 4

| | |
|---|---|
| R02248 | MAMAG |
| R08440 | AAAG |

The binding site R08440 is covered by the other R02248. In TRANSFAC database, 3906 binding sites belong to this type. Each site may or may not have transcription factor names. 3006 accession numbers have transcription factor names.

### EXAMPLE 5

| | |
|---|---|
| R00001 | ISGF-3 |
| R00002 | ICSBP |
| R00003 | ISGF-3 |
| R00303 | Oct-1C Oct-1B Oct-4 Oct-1A |
| R00304 | Oct-4 Oct-1A Oct-1B Oct-1C |
| R00305 | Oct-4 Oct-1A Oct-1B Oct-1C |
| R00306 | Oct-1B Oct-1C Oct-4 Oct-1A |

Example 5 shows another situation. Different binding sites contain the same set of transcription factor names. For example, the binding sites R00303, R00304, R00305, R00306 have the same transcription factor names, i.e., Oct-1C Oct-1B Oct-4 Oct-1A.

Significance Level

The significance level measurement classifying correlated and independent is defined herein as followings:

Definition 1 (correlated): Where s is a minimum support, t is a significance level, A is a set of items and B is an item. Assume that the rule A=>B is correlated if it satisfies the following two conditions:

(1). The support exceeds s.

(2). The significance level exceeds t.

Definition 2 (independent): Let s be a minimum support, t be a significance level, A be a set of items, and B be an item. Assume that the rule A=>B is independent if it satisfies the following two conditions.

(1). The support exceeds s.

(2). The significance level does not exceed t.

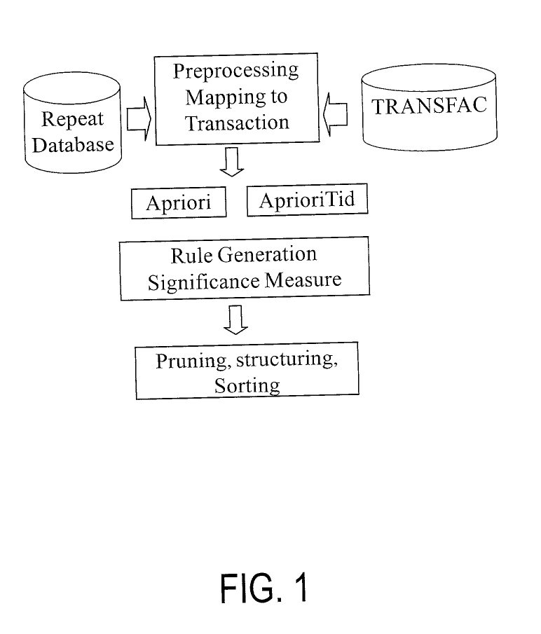

FIG. 1 illustrates the proposed approach according to one preferred embodiment of the invention. A preprocessing process, including mapping between the transcription factor binding sites in TRANSFAC and the repeat sequences in the repeat sequence database, is applied. Next, data mining approach, such as Apriori and AprioriTid, are applied to mine the transaction rules by combining the transcription factor binding sites in the repeat sequences. The Apriori and AprioriTid algorithms are focused in finding all common patterns embedded in a database of sequences of sets of events. The input data of such mining approach is a set of sequences, called data-sequences. Each data-sequence is a list of transactions, where each transaction is a set of characters (literals), called items. A sequential pattern also consists of a list of sets of items. The approach is to find all sequential patterns with a user-specified minimum support, where the support of a sequential pattern is the percentage of data-sequences that contain the pattern. The significance test, such as Chi-square, is used to select certain rules. Later on, redundant rules are pruned and structured.

Steps of the proposed approach are summarized as follows:

(1) Determine the number of item sets of the transcription factor binding sites in TRANSFAC.

(2) For categorical binding sites, identification of a binding site is mapped to a set of transcription factor names.

(3) Find the combinations of transcription factors in the repeat sequences.

(4) Apply the data mining approach to generate association rules.

(5) Determine the interesting rules using the Chi-square significance test.

(6) Prune redundant rules.

(7) Classify rules to cover and non-cover sets.

(8) Partially classify repeat sequences by using association rules that are previously mined.

Preprocessing and Mapping between the Data in the Repeat Sequence Database and in TRANSFAC Database

The transcription factor binding sites in TRANSFAC database above are first prepared due to the complicated situations described above. This accounts for why the proposed approach requires preprocessing. Combinations of the transcription factor binding sites in the repeat sequences in our repeat sequence database are then found. This work focuses mainly on the repeat sequences of the genomes C. Elegans, Human Chromosome 22, Yeast, and several bacteria. Table 1 summarizes the results of the preprocessing. The abbreviation of the organisms in Table 1 is given in Appendix A.

TABLE 1
Combinations of transcription factor binding sites
for C. Elegans, Human Chromosome 22, Yeast, archaea,
bacteria, and virus.

| | Total | | | More | | |
|---|---|---|---|---|---|---|
| | Repeat | | | Than | | |
| Genome | Sequ- | Match | No | One | Average | |
| Name | ences | One | Match | Match | Factors | Ratio |
| C. | 454927 | 73881 | 29962 | 351084 | 4.8 | 77.17% |
| Elegans | | | | | | |
| Human | 1347364 | 47159 | 22211 | 1277994 | 7.6 | 94.85% |
| Chromo- | | | | | | |
| some 22 | | | | | | |
| Yeast | 4329 | 305 | 338 | 3686 | 22.5 | 85.14% |
| Bsub | 700 | 73 | 27 | 600 | 11.5 | 85.71% |
| Hinf | 788 | 93 | 55 | 640 | 7.3 | 81.22% |
| Hpyl | 713 | 98 | 25 | 590 | 8.3 | 82.75% |
| Hpy199 | 721 | 88 | 33 | 600 | 6.3 | 83.22% |
| Mgen | 373 | 26 | 16 | 331 | 6.7 | 88.74% |
| Mtub | 4932 | 784 | 171 | 3977 | 5.1 | 80.64% |
| E coli | 1897 | 188 | 60 | 1649 | 8.8 | 86.93% |
| CP | 135 | 14 | 8 | 113 | 7.3 | 83.70% |
| MP | 1282 | 107 | 36 | 1139 | 7.5 | 88.85% |
| RP | 98 | 8 | 2 | 88 | 5.8 | 89.80% |
| TP | 102 | 7 | 4 | 91 | 15.3 | 89.22% |
| AP | 398 | 62 | 7 | 329 | 7.4 | 82.66% |
| AR | 779 | 48 | 21 | 710 | 7.8 | 91.42% |
| PA | 277 | 20 | 4 | 253 | 5.1 | 91.34% |
| PH | 401 | 17 | 4 | 380 | 6.5 | 94.76% |
| AA | 299 | 20 | 7 | 272 | 6.9 | 90.97% |
| CT | 27 | 4 | 1 | 22 | 14.5 | 81.48% |
| S | 1580 | 78 | 34 | 1468 | 9.1 | 92.91% |
| TM | 518 | 24 | 14 | 480 | 7.0 | 92.66% |
| UU | 302 | 31 | 9 | 262 | 6.2 | 86.75% |

Each row refers to a genome or bacteria that is experimented with. The column “Average Factors” represents the average transcription factor binding sites found in a repeat sequence, As mentioned above, we find the combinations of transcription factors in repeat sequences. The “Average Factors” is defined to be the sum of the transcription factor binding sites for all repetitive sequences over the sum of the repetitive sequences. The last column “Ratio” denotes the number of repetitive sequences containing more than one binding site over the total repetitive sequences in a genome. For example, the ratio 77.17% in C. Elegans indicates 77.17% repeat sequences, i.e. 351,084 ones that will be used to mine associations.

Exactly how to mine associations from the combinations of the transcription factor binding sites found above is discussed as follows. Consider a large database with transactions, where each transaction consists of a set of items. An association rule can be expressed as A=>B, where A and B are the sets of items. The mining of an association rule is to find a transaction that contains A and tends to contain B in the database. For example, 90% of the people who purchase beer also purchase diapers. Herein, 90% is called the confidence of the rule. The support of the rule A=>B given herein is the percentage of transactions that contain both A and B.

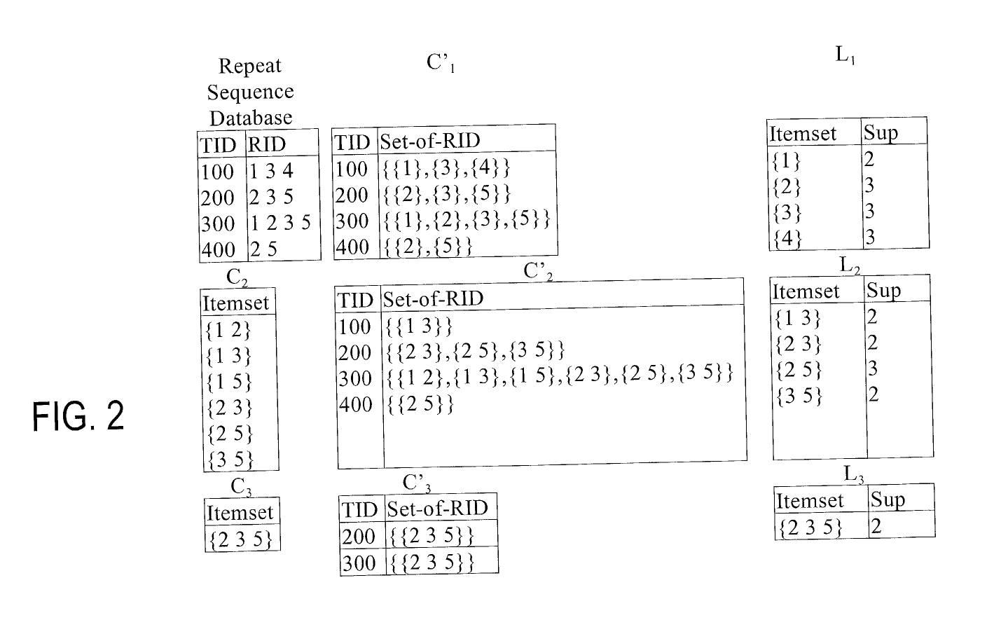

The formal statement of the problem is described below. Let I={i₁, i₂, . . . , iₘ} be a set of sites, called item set. Let D be a set of repeat sequences, where each repeat sequence S corresponding to a transaction contains a set of items such that S⊂I . FIG. 2 presents an example of mapping the repeat sequences and transcription factor binding sites, where TID is a number of a repetitive sequences and RID is a set of IDs of binding sites. In the proposed approach, only consider repetitive sequences that contain more than one binding site.

Example 6 illustrates the mapping between a repeat sequence and the transcription factor binding sites.

### EXAMPLE 6

>IDI0000000013
AGTTATTCAAACACGTATAA
TTCAAA
R02749
TATAA
R00046 R00705 R00706 R03054
TATA
R00671 R00689 R00938 R01128 R01129 R01191 R04293

In Example 6, “AGTTATTCAAACACGTATAA” is a repeat sequence in the repeat sequence database. We map it to a transaction whose id is IDI0000000013. The repeat sequence has three consensus patterns, i.e., “TTCAAA”, “TATAA”, and “TATA”. The consensus pattern “TTCAAA” has an accession number R02749. However, the other two consensus patterns “TATAA” and “TATA” have many accession numbers. For this kind of situation, the preprocessing process is required. Example 7 is another case. Similarly, IDI0000000737 is a transaction ID mapped from a repeat sequence “TTGAAATTTTGAAATTTAAA”. The repeat sequence has four consensus patterns.

### EXAMPLE 7

| | |
|---|---|
| >IDI0000000737 | |
| TTGAAATTTTGAAATTTAAA | |
| TTGAA | R04347 R04360 R04369 |
| ATTTNNNNATTT | R02171 |
| TKINNGNAAK | R02216 |
| TTTAAA | R01598 |

Example 7 presents the results after the mapping. Each list shows the factor name, consensus sequences and the identification of the binding site.

### EXAMPLE 8

>IDI0000000737
TTGAAATTTTGAAATTTAAA
DE unknown = TTTAAA>R01598
DE unknown = TTGAA>R04347\R04360\R04369
DE HiNF-A = ATTTNNNNATTT>R02171
DE C/EBPbeta\C/EBPdelta = TKNNGNAAK>R02216

In Example 8, repeat sequence (transaction) “TTGAAATTTTGAAATTTAAA” contains four consensus patterns (items), i.e., TTTAAA, TTGAA, ATTTNNNNATTT, and TKNNGNAAK. Example 8 lists different possible situations, as described below.

(1) One site and no factor: They resemble R01598.

(2) One site and one factor: They resemble R02171 with the factor HiNF-A.

(3) One site with many accession numbers: It is like R04347, R04360, and R04369 with the same consensus sequence TTGAA.

(4) One site and many factors: They resemble R02216 with factors “C/EBPbeta” and “C/EBPdelta”. Different factors or binding sites are separated by the symbol “\” . A transaction and its containing items can be expressed as Example 9 below.

### EXAMPLE 9

>IDI0000000737 R04347\R04360\R04369 HiNF-A C/EBPdelta\C/EBPbeta R01598

In Example 9, the transaction IDI)0000000737 contains four items that are denoted R04347\R04360\R04369, HiNF-A, C/EBPdelta\C/EBPbeta, and R01598, respectively.

Assume that a repeat sequence S contains A, a set of items of I, if A⊂S. An association rule is an inference of the form A=>B, where A⊂I, B⊂I, and A∩B =0.

The rule A=>B holds in the repetitive sequence set D with confidence (conf) c if c% of transactions in D contains A and also B. The rule A=>B has support (sup) s in the repetitive sequence set D if s% of repeat sequences in D contained A∪B our experiments, the minimum support is set to 10%. The association rules are generated if the rule has a higher support and confidence than user specified. Data mining approaches, such as Apriori and AprioriTid, are then applied to mine association rules.

An enormous number of association rules are generated. The enormous number of association rules makes it extremely difficult for human users to identify those interesting and useful ones. Therefore, Chi-square is applied to prune the discovered association rules in order to remove those insignificant association rules. Pruning and structuring association results

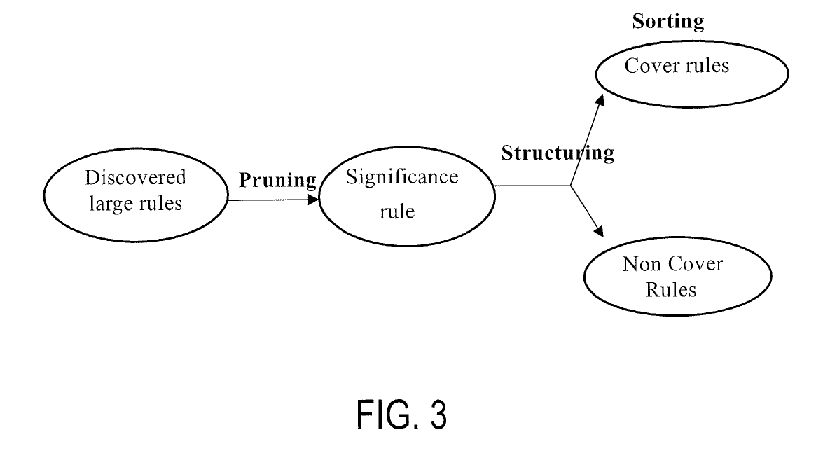

Herein, rules are generated using the Chi-square significance test. The discovered rules are still large and unreadable after applying the process of Chi-square significance test. The redundant rules are pruned and the remained rules are structured to cover set and non-cover set. FIG. 3 presents the conceptual flow of the pruning and structuring. Firstly, discovered rules may be not significant for several reasons. Rules corresponding to either the prior biology knowledge or certain expectations are in main interests. Secondly, rules can refer to non-interested sites or sites combinations such as transcription factor binding sites on protein to C. Elegans. Thirdly, rules can be redundant.

Three operations are used to process a large collection of rules.

1. Pruning: reduce the insignificant rules.

2. Structuring: divide the rules into cover and non-cover sets.

3. Sorting: rank the rules by the use of confidence.

The Chi-square significance test ignores simple redundancy and strict redundancy. For example, the rule AB=>C is redundant to A=>BC. The rule AB=>C is tested, while A=>BC is not. The strict rule A=>B is redundant to A=>BC, and A=>B is tested. The redundancy of our rules is similarly determined. The rule A=>B is kept and the rule AC=>B is pruned because AC=>B is covered by the rule A=>B. For example, consider the rule MAMAG=>AAAG. Obviously, the binding site on the right-hand side is covered by that on the left-hand side because M may be A or C. The rule is put into the cover set. Tables 2 and 3 present the association rules mined after applying the Chi-square test from Table 1. In Table 3, the significance level is set to 95%. In Table 2, the “MiniSup” column refers to the minimum support used. The “Cover Rules” and “Non Cover Rules” denote the number of rules in the cover and non-10 cover sets, respectively, after they are mined, pruned, and structured. The “Total Rules” denotes the sum the rules in the cover and non-cover sets. The “Ratio of Partial Classification” represents the ratio of the repeat sequences are classified by the “Total Rules”. For example, 47% repeat sequences of C. Elegans are partially classified by the ten mined rules. Conversely, it indicates that the other 53% repeat sequences cannot be classified by the rules. Therefore, the ratio can also be used to measure whether the mined rules are representative. Similarly, Table 3 summarizes the data for archaea, bacteria, and virus. The minimum support is set to 10% and those with the “*” symbol in the precedence of the genome name is set to 20%.

TABLE 2
The association rules mined after applying the Chi-square test.

| | | | | | Ratio of |
|---|---|---|---|---|---|
| | | | Non | | Partial |
| | | Cover | Cover | Total | Classifi- |
| Genome Name | MiniSup | Rules | Rules | Rules | cation |
| C. Elegans | 5% | 4 | 6 | 10 | 47% |
| Human | 28% | 4 | 6 | 10 | 79% |
| Chromosome 22 | | | | | |
| Yeast | 31% | 5 | 5 | 10 | 77% |

TABLE 3
The association rules for archaea, bacteria and virus are
mined after applying the Chi-square test.

| | Prune | Non | | Total |
|---|---|---|---|---|
| Genome Name | Rules | Cover Rules | Cover Rules | Rules |
| Bsub | 63 | 103 | 55 | 158 |
| Hinf | 3 | 3 | 3 | 6 |
| Hpyl | 0 | 3 | 1 | 4 |
| Hpy199 | 18 | 11 | 21 | 32 |
| Mgen | 19 | 17 | 11 | 28 |
| Mtub | 0 | 5 | 1 | 6 |
| E coli | 0 | 1 | 1 | 2 |
| CP | 0 | 3 | 1 | 4 |
| MP | 0 | 3 | 5 | 8 |
| RP | 3 | 10 | 14 | 24 |
| *TP | 0 | 8 | 10 | 18 |
| AP | 31 | 24 | 26 | 50 |
| AR | 1004 | 74 | 15 | 89 |
| PA | 3 | 4 | 2 | 6 |
| PH | 55 | 8 | 12 | 20 |
| AA | 0 | 3 | 5 | 8 |
| *CT | 0 | 4 | 2 | 6 |
| S | 3 | 22 | 18 | 40 |
| TM | 55 | 20 | 6 | 26 |
| UU | 0 | 8 | 8 | 16 |

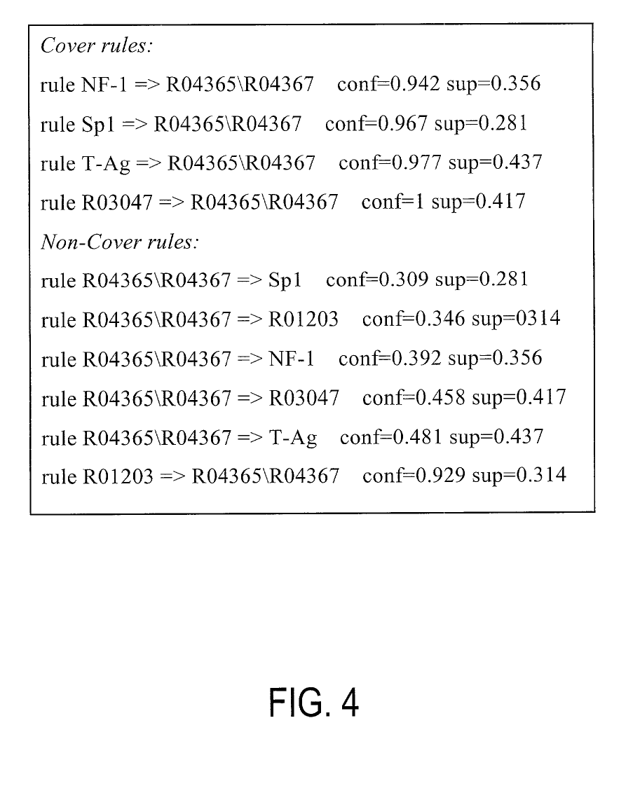

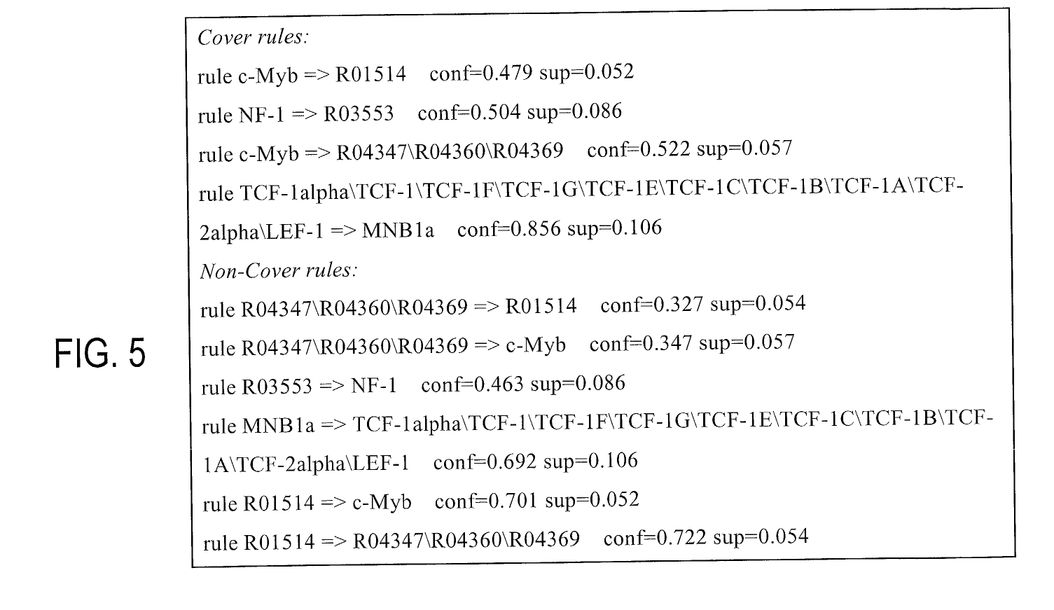

FIGS. 4 and 5 present partial classification rules for the Human Chromosome 22 and C. Elegans Genome, respectively. These rules can be used to find genes in complete genomes and cluster repeat sequences once they are verified.

To verify the association rules found in repetitive sequences also appear in their genomes, further experiments are applied on archaea and bacteria because of their shorter genome sizes. The experimental results are shown in Table 4. The column “Occurrences in Repeats” denotes how many copies of a repetitive sequence are found in a genome. The column “Occurrences in Genome” represents how many associations are found in a genome. The “Window” column indicates the offset of the transcription factors binding site, e.g., the difference of the transcription factors binding site. For example, two of the rules YY1=••• and YY1=>••• are found in a repetitive sequence of the organism Pyrococcus abyssi. Please refer to Appendix B for more details of the two rules. The repetitive copies of the repetitive sequence are 39. We then go back to its genome scale and find the association YY1=R00388 also exist in 48 different positions when the window is set 5. The larger of the window is, the more associations are found. However, a huge amount of associations are found in a genome scale such as Thermotoga maritima even the occurrences of the repetitive sequence is not large.

TABLE 4
The association rules in a small scale (repetitive sequences) and genome scale.

| | | Occurrences | Occurrences in Genome | | |
|---|---|---|---|---|---|
| Organism | Association Rules | in Repeats | Window = 1 | Window = 5 | Window = 10 |
| Thermotoga | c-Ets-2=>R03553 | 272 | 1506 | 1700 | 2019 |
| maritima | R03553=>R01230 | 220 | 0 | 56 | 332 |
| | c-Ets-2=>R01230 | 218 | 0 | 66 | 206 |
| Mycoplasma | TCF-1alpha\TCF-1\TCF-1F\TCF- | 208 | 3785 | 3954 | 4557 |
| genitalium | 1G\TCF-1E\TCF-1C\TCF-1B\TCF- | | | | |
| | 1A\TCF-2alpha\LEF-1=>MNB1a | | | | |
| Treponema | Spl=>R03047 | 33 | 549 | 719 | 1219 |
| pallidum subsp. | | | | | |
| Pallidum | | | | | |
| | Spl=>T-Ag | 39 | 984 | 1285 | 1779 |
| | Spl=>GAL4 | 39 | 474 | 1150 | 1883 |
| | GAL4=>R04141 | 39 | 0 | 1641 | 1853 |
| | R01203=>R04398 | 33 | 0 | 602 | 817 |
| | GAL4=>R03047 | 39 | 0 | 161 | 416 |
| | R04398=>R00290\R01241\R01244 | 43 | 879 | 894 | 940 |
| Ureaplasma | YY1=>R01513 | 62 | 754 | 2003 | 2614 |
| urealyticum | YY1=>Pit-1a | 60 | 0 | 893 | 1859 |
| | N-Oct-3=>Pit-1a | 64 | 179 | 2610 | 3230 |
| | TCF-1alpha\TCF-1\TCF-1F\TCF- | 72 | 3202 | 3295 | 3650 |
| | 1G\TCF-1E\TCF-1C\TCF-1B\TCF- | | | | |
| | 1A\TCF-2alpha\LEF-1=>MNB1a | | | | |
| | Pit-1a=>R01598 | 50 | 0 | 1305 | 1621 |
| | Pit-1a=>YY1 | 60 | 0 | 893 | 1859 |
| | R01513=>YY1 | 62 | 754 | 2003 | 2614 |
| Pyrococcus | YY1=>R00231\R00232\R00335\ | 39 | 0 | 34 | 105 |
| abyssi | R00668\R00669\R00761\R01081\ | | | | |
| | R01345\R01445\R01446\R02955\R02957 | | | | |
| | YY1 =>R00388 | 41 | 0 | 48 | 175 |
| | R00388=>R00231\R00232\R00335\ | 37 | 0 | 37 | 64 |
| | R00668\R00669\R00761\R01081\ | | | | |
| | R01345\R01445\R01446\R02955\ | | | | |
| | R02957 | | | | |
| Synechocystis | NF-1=>R03553 | 356 | 6328 | 9307 | 12568 |
| PCC6803 | | | | | |
| | TCF-1alpha\TCF-1\TCF-1F\TCF- | 449 | 12871 | 13209 | 14597 |
| | 1G\TCF-1E\TCF-1C\TCF-1B\TCF- | | | | |
| | 1A\TCF-2alpha\LEF-1=>MNB1a | | | | |
| | NF-1=>R00291 | 469 | 696 | 3506 | 5305 |
| Rickettsia | YY1=>TFIID | 16 | 335 | 551 | 975 |
| prowazekii | N-Oct-3=>ETF | 14 | 445 | 1334 | 1728 |
| | YY1=>SEF4 | 22 | 872 | 1017 | 1275 |
| | YY1=>R01513 | 24 | 1024 | 2265 | 3051 |
| | Pit-1a=>N-Oct-3 | 18 | 111 | 2571 | 2991 |
| | R00671\R00689\R00938\R01128\ | 14 | 2037 | 2382 | 2869 |
| | R01129\R01191\R04293=>TFIID | | | | |
| | R00671\R00689\R00938\R01128\ | 16 | 4769 | 5071 | 5716 |
| | R01129\R01191\R04293=>R00583 | | | | |
| | R00671\R00689\R00938\R01128\ | 18 | 0 | 2519 | 3374 |
| | R01129\R01191\R04293=>R01513 | | | | |
| | Pit-1a=>R01598 | 18 | 0 | 869 | 1035 |
| | ETF=>TFIID | 14 | 2724 | 2754 | 2982 |

This study finds combinations of transcription factor binding sites in the repeat sequences in the repeat sequence database. Each repeat sequence is mapped to a transaction and combinations of transcription factor binding sites are mapped to items of a transaction. The transcription factor binding sites in TRANSFAC database need to be preprocessed due to their complex characteristics. The data mining approaches are then applied to mine the associations from the combinations of transcription factor binding sites in repeat sequences. An enormous number of association rules are generated. The Chi-square significance level is used to remove those insignificant rules. The association rules are pruned, structured and sorted into cover and non-cover sets. Moreover, experiments are conducted on many genomes including C. Elegans, Human Chromosome 22, Yeast, and bacteria. The mined rules can also be used to find useful genes in complete genomes as well as partially cluster the repeat sequences in the repeat sequence database.

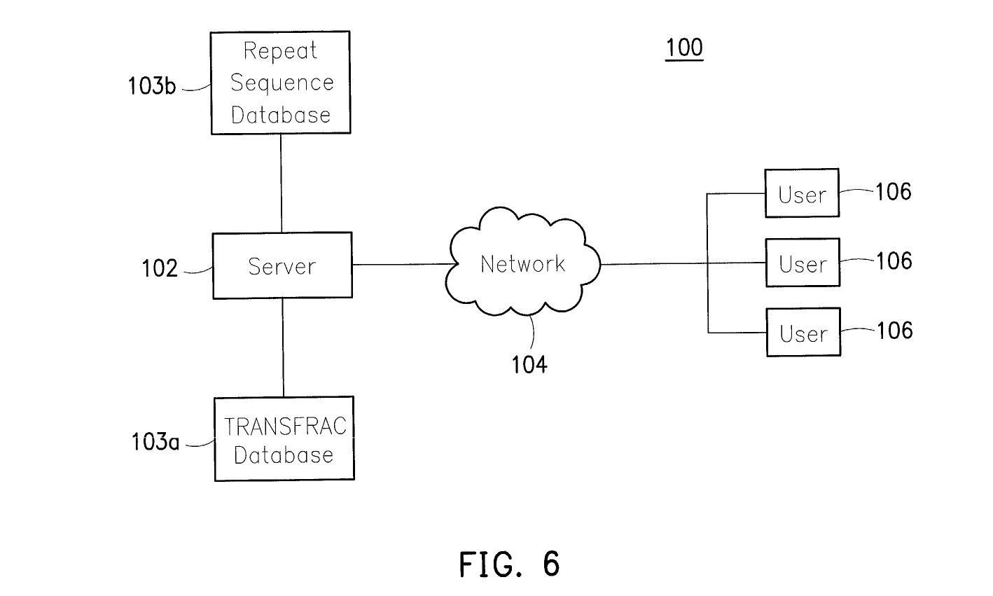

The method of the present invention, as described in the previous sections, can be used in a computerized system for mining association rules from combinations of transcription factor binding sites in repeat sequences and for further predicting regulatory elements in repetitive sequences using transcription factor binding sites. As shown in FIG. 6, the computerized system 100 that applies the method for mining association rules can be an open system including a server 102. The server 102 is accessible over a computer network 104 by other authorized users 106 for either providing initial data resources or inputting commands. The server 102 includes means for storing. The server 102 can assess various databases, such as a TRANSFRAC database 103a and/or a repeat sequence database 103b, to acquire data resources. The server 102 further includes means for preprocessing the acquired data resources. The server 102 can output the final data resources over the computer network 104 back to the authorized users 106 based on the commands. The means for transferring the data resources and the commands (either inputting or outputting) can be, for example, TC/PIP. However, every possible means for transferring the data resources and the commands available at the time is within the scope of the invention. On the other hand, the computerized system can be a close system running the method of the present invention.

Furthermore, the method of predicting regulatory elements in the repetitive sequences can be configured as a computer readable program. Persons skilled in the relevant art will be able to produce such computer readable program based on the discussion of the proposed method contained herein.

The exemplary embodiments have been primarily described with reference to flow charts illustrating pertinent features of the embodiments. Each method step may also represent a hardware or software component for performing the corresponding step. It should be appreciated that not all components or method steps of a complete implementation of a practical system are necessarily illustrated or described in detail. Rather, only those components or method steps necessary for a thorough understanding of the invention have been illustrated and described in detail. Actual implementations may utilize more steps or components or fewer steps or components.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

[image]

[image]

What is claimed is:

1. A method for predicting regulatory elements in repetitive sequences using transcription factor binding sites, comprising:
preprocessing the transcription factor binding sites in a transcription factor binding site database and mapping the transcription factor binding sites to transcription factor names;
mapping the transcription factor binding sites in the transcription factor binding site database to repeat sequences in a repeat sequence database in order to find combinations of transcription factors in the repeat sequences;
applying a data mining approach to generate association rules;
pruning a portion of the generated association rules by using a significance test;
classifying the remained association rules to cover and non-cover sets after pruning; and
using the remained association rules to classify the repeat sequences in the repeat sequence database.

2. The method as claimed in claim 1, wherein the transcription factor binding site database comprises a TRANSFAC database.

3. The method as claimed in claim 1, wherein the significance test comprises a Chi-square test.

4. The method as claimed in claim 1, wherein the step of applying the data mining approach comprises the following steps:
inputting a set of data-sequences, wherein each data-sequence is a list of transactions and each transaction is a set of items;
providing a plurality of sequential patterns, wherein each sequential pattern consists of a list of sets of items; and
finding the sequential patterns with a user-specified minimum support in the data-sequences, where the support of a sequential pattern is a percentage of data-sequences that contain the pattern.

5. A method for mining association rules from combinations of transcription factor binding sites in repeat sequences, comprising:
preprocessing the transcription factor binding sites in a transcription factor binding site database and mapping the transcription factor binding sites to transcription factor names;
mapping the transcription factor binding sites in the transcription factor binding site database to repeat sequences in a repeat sequence database in order to find the combinations of transcription factors in the repeat sequences;
applying a data mining approach to generate association rules;
using a significance test to prune a portion of the association rules; and
classifying the remained association rules to cover and non-cover sets.

6. The method as claimed in claim 5, wherein the transcription factor binding site database comprises a TRANSFAC database.

7. The method as claimed in claim 5, wherein the significance test comprises a Chi-square test.

8. The method as claimed in claim 5, wherein the step of applying the data mining approach comprises the following steps:
inputting a set of data-sequences, wherein each data-sequence is a list of transactions and each transaction is a set of items;
providing a plurality of sequential patterns, wherein each sequential pattern consists of a list of sets of items; and
finding the sequential patterns with a user-specified minimum support in the data-sequences, where the support of a sequential pattern is a percentage of data-sequences that contain the pattern..

9. A computerized system for predicting regulatory elements in repetitive sequences using transcription factor binding sites, wherein the system can assess the transcription factor binding site database and a repeat sequence database, the system comprising:
means for inputting commands from a user;
means for storing;
means for preprocessing the transcription factor binding sites in the transcription factor binding site database and mapping the transcription factor binding sites to transcription factor names;
means for mapping the transcription factor binding sites in the transcription factor binding site database to repeat sequences in the repeat sequence database, in order to find the combinations of transcription factors in the repeat sequences;
means for generating association rules by applying a data mining approach;
means for pruning a portion of the mined association rules using a significance test;
means for classifying the remained association rules to cover and non-cover sets;
means for classifying the repeat sequences in the repeat sequence database using the mined association rules; and
means for outputting.

10. The system as claimed in claim 10, wherein the transcription factor binding site database comprises a TRANSFAC database.

11. The method as claimed in claim 10, wherein the significance test comprises a Chi-square test.

12. The method as claimed in claim 10, wherein the data mining approach comprises the following steps:
inputting a set of data-sequences, wherein each data-sequence is a list of transactions and each transaction is a set of items;
providing a plurality of sequential patterns, wherein each sequential pattern consists of a list of sets of items; and
finding the sequential patterns with a user-specified minimum support in the data-sequences, where the support of a sequential pattern is a percentage of data-sequences that contain the pattern.

13. A storage system comprising an operating program for predicting regulatory elements in repetitive sequences using transcription factor binding sites, wherein the program comprises instructions for causing the system to:
preprocess the transcription factor binding sites in a transcription factor binding site database and mapping the transcription factor binding sites to transcription factor names;
map the transcription factor binding sites in the transcription factor binding site database to repeat sequences in a repeat sequence database in order to find combinations of transcription factors in the repeat sequences;
apply a data mining approach to generate association rules;
prune a portion of the generated association rules by using a significance test;
classify the remained association rules to cover and non-cover sets after pruning; and
classify the repeat sequences in the repeat sequence database using the remained association rules.

14. The system as claimed in claim 13, wherein the transcription factor binding site database comprises a TRANSFAC database.

15. The method as claimed in claim 13, wherein the significance test comprises a Chi-square test.

16. The method as claimed in claim 13, wherein the application of the data mining approach comprises the following steps:
inputting a set of data-sequences, wherein each data-sequence is a list of transactions and each transaction is a set of items;
providing a plurality of sequential patterns, wherein each sequential pattern consists of a list of sets of items; and
finding the sequential patterns with a user-specified minimum support in the data-sequences, where the support of a sequential pattern is a percentage of data-sequences that contain the pattern.

17. A storage system comprising an operating program for mining association rules from combinations of transcription factor binding sites in repeat sequences, wherein the program comprises instructions for causing the system to:
preprocess the transcription factor binding sites in a transcription factor binding site database and mapping the transcription factor binding sites to transcription factor names;
map the transcription factor binding sites in the transcription factor binding site database to repeat sequences in a repeat sequence database in order to find combinations of transcription factors in the repeat sequences;
apply a data mining approach to generate association rules;
use a significance test to prune a portion of the generated association rules; and
classify the remained association rules to cover and non-cover sets.

18. The system as claimed in claim 17, wherein the transcription factor binding site database comprises a TRANSFAC database.

19. The method as claimed in claim 17, wherein the significance test comprises a Chi-square test.

20. The method as claimed in claim 17, wherein the application of the data mining approach comprises the following steps:
inputting a set of data-sequences, wherein each data-sequence is a list of transactions and each transaction is a set of items;
providing a plurality of sequential patterns, wherein each sequential pattern consists of a list of sets of items; and
finding the sequential patterns with a user-specified minimum support in the data-sequences, where the support of a sequential pattern is a percentage of data-sequences that contain the pattern.
